(19) United States
(12) Patent Application Publication
(10) Pub. No.: US 2008/0145422 A1
(43) Pub. Date: Jun. 19, 2008

(54) Galantamine tablet formulation

(75) Inventors: Yunlong Zhou, Dublin, OH (US); Andrea McPhillips, Dublin, OH (US); Julie Economou, Dublin, OH (US)

Correspondence Address: MICHAEL P. MORRIS;BOEHRINGER INGELHEIM CORPORATION, 900 RIDGEBURY ROAD, P O BOX 368, RIDGEFIELD, CT, 06877-0368, US

(73) Assignee: Roxane Laboratories, Inc., Columbus, OH (US)

(21) Appl. No.: 11/055,189

(22) Filed: Feb. 10, 2005

(51) Int. Cl. A61K 31/55; A61K 9/20

(52) U.S. Cl. 424/464; 514/214.01

(57) ABSTRACT

The present invention relates to a direct compression tablet formulation comprising, as the active ingredient, galantamine, and more specifically, galantamine hydrobromide along with a process of making the same. The tablet formulation is a direct compression tablet and has excellent content uniformity and dissolution properties.

### FIELD OF THE INVENTION

The present invention relates to a tablet formulation comprising, as the active ingredient, galantamine, preferably, galantamine hydrobromide. The tablet formulation is a direct compression tablet and has excellent content uniformity and dissolution properties.

### BACKGROUND OF THE INVENTION

Galantamine is an oral medication used to treat patients with Alzheimer's disease. Galantamine is in a class of drugs called cholinesterase inhibitors that also includes tacrine (Cognex), donezepil (Aricept), and rivastigmine (Exelon). Cholinesterase inhibitors inhibit (block) the action of acetylcholinesterase, the enzyme responsible for the destruction of acetylcholine. Acetylcholine is one of several neurotransmitters in the brain, chemicals that nerve cells use to communicate with one another. Reduced levels of acetylcholine in the brain are believed to be responsible for some of the symptoms of Alzheimer's disease. By blocking the enzyme that destroys acetylcholine, galantamine increases the concentration of acetylcholine in the brain, and this increase is believed to be responsible for the improvement in cognitive ability seen with galantamine.

The use of galantamine or an analogue or a pharmaceutically acceptable acid addition salt thereof for the preparation of a medicament for treating Alzheimer's Dementia (AD) and related dementias has been described in EP-0,236,684 (U.S. Pat. No. 4,663,318).

The use of galantamine for treating alcoholism and the administration via a transdermal transport system (TTS) or patch is disclosed in EP-0,449,247. Similarly, the use of galantamine in the treatment of nicotine dependence using administration via a transdermal transport system (TTS) or patch is disclosed in WO-94/16708.

A number of U.S. and foreign patents disclose the use of galantamine, analogues thereof and pharmaceutically acceptable salts thereof for the preparation of medicaments for treating mania (U.S. Pat. No. 5,336,675), chronic fatigue syndrome (CFS) (FP-0,515,302 ; U.S. Pat. No. 5,312,817), and the negative effects of benzodiazepine treatment (EP-0,515,301). In U.S. Pat. No. 5,312,817, a number of specific tablet formulations of galantamine hydrobromide are given.

Traditionally, galantamine hydrobromide was administered as an injectable or as an oral solution. Although tablet formulations have been described in the prior art (U.S. Pat. No. 5,312,817), problems with direct compression tableting have been recognized. In U.S. Pat. No. 6,358,527, problems relating to segregation of ingredients (see column 3, lines 13-20 of U.S. Pat. No. 6,358,527) and slow dissolution (see column 3, lines 20-22 of U.S. Pat. No. 6,358,527) were described when direct compression of galantamine hydrobromide was attempted using either lactose anhydrous or lactose monohydrate as a diluent and either powdered cellulose or microcrystalline cellulose as a disintegrant.

The present invention relates to a novel formulation for delivering galantamine to a patient in need thereof. In particular, the formulation is a direct compression tablet of galantamine hydrobromide which has excellent content uniformity along with excellent dissolution properties.

### SUMMARY OF THE INVENTION

In accordance with the invention, a pharmaceutical tablet composition comprising, as an active ingredient, galantamine hydrobromide and a pharmaceutically acceptable carrier is provided, wherein said pharmaceutically acceptable carrier comprises a direct blend of a lactose-based diluent and microcrystalline cellulose.

More specifically, a pharmaceutical tablet composition comprising, as an active ingredient, galantamine hydrobromide and a pharmaceutically acceptable carrier is provided, wherein said pharmaceutically acceptable carrier comprises a direct blend of lactose anhydrous and microcrystalline cellulose.

In each of the above tablet compositions, additional pharmaceutically acceptable formulation adjuvants may be incorporated into the tablet compositions. Such pharmaceutically acceptable formulation adjuvants include, but are not necessarily limited to, one or more disintegrants, glidants, lubricants, and coating agents.

Another embodiment of the invention comprises a pharmaceutical, direct compression tablet composition comprising:
(a) from about 2% to about 10% (w/w) galantamine hydrobromide;
(b) from about 1% to about 95% (w/w) lactose-based diluent;
(c) from about 1% to about 95% (w/w) microcrystalline cellulose;
(d) from about 0% to about 10% (w/w) disintegrant;
(e) from about 0.1% to about 2% (w/w) glidant;
(f) from about 0.1% to about 2% (w/w) lubricant.

More preferably, according to the above embodiment, the lactose-based diluent is lactose anhydrous, the disintegrant is crospovidone, the glidant is colloidal silicon dioxide, and the lubricant is magnesium stearate. A pharmaceutically acceptable coating may also be present in the tablet formulations according to the invention.

In addition to be above, the present invention also includes a process of making a direct compression tablet comprising galantamine, and more particularly galantamine hydrobromide, as the active ingredient. The direct compression galantamine tablet can be formed by a direct compression tableting process comprising the steps of:
(a) passing galantamine hydrobromide, a lactose-based diluent, microcrystalline cellulose, a disintegrant, and a glidant through a delumping device to form a first delumped material;
(b) blending the first delumped material of step (a) to form a first blend;
(c) passing a lubricant through a delumping device;
(d) blending the delumped lubricant of step (c) with the first blend of step (b) to form a second blend;
(e) compressing the second blend into a direct compression tablet.

### A coating step may follow the compression step wherein a pharmaceutically acceptable coating agent is applied to the compression formed tablet.

The galantamine hydrobromide tablets formed according to the invention have excellent content uniformity along with excellent dissolution properties.

### DETAILED DESCRIPTION OF THE INVENTION

The present invention relates to a direct compression tablet formulation comprising galantamine and its pharmaceutically acceptable salts, and in particular galantamine hydrobromide, as the active ingredient. The galantamine tablets of the present invention have excellent content uniformity along with excellent dissolution properties.

According to the present invention, galantamine is able to be formulated into a tablet by a direct compression method. The tablets of the invention are suitable as unit dosage forms. The tablets contain a therapeutically effective amount of galantamine. One aspect of the invention is directed to direct compression tablets of galantamine hydrobromide which contain from about 4 mg to about 16 mg of galantamine hydrobromide. In more preferred embodiments, direct compression tablets containing 4 mg, 8 mg, and 12 mg dosage amounts of galantamine hydrobromide are produced according to the invention.

Treatment regimens which utilize galantamine and its salts include those conventional in the art. In particular, treatment regimens for Alzheimer's disease include, but are not limited to, those described in U.S. Pat. No. 4,663,318, the disclosure of which is incorporated herein by reference.

In accordance with the present invention, tablets comprising galantamine, and in particular galantamine hydrobromide, are prepared via a direct compression method. Various pharmaceutically acceptable tablet formulation adjuvants are also included in the formulations according to the present invention. Such pharmaceutically acceptable tablet formulation adjuvants include, but are not necessarily limited to, diluents, disintegrants, lubricants, glidants, and coating agents.

According to one aspect of the invention, a pharmaceutical tablet composition comprising, as an active ingredient, galantamine hydrobromide, and a pharmaceutically acceptable carrier is formed. The pharmaceutically acceptable carrier comprises a direct blend of a lactose-based diluent and microcrystalline cellulose.

In accordance with a further aspect of the invention, galantamine hydrobromide is formulated with a pharmaceutically acceptable carrier which includes, but is not necessarily limited to, a direct blend of a lactose-based diluent and microcrystalline cellulose along with one or more disintegrants, glidants, lubricants, and coating agents.

Pharmaceutically acceptable lactose-based diluents include, but are not limited to, one or more of lactose anhydrous, lactose monohydrate, and spray-dried lactose. The lactose-based diluents may be used alone or in combination. The lactose-based diluent can be present in amounts ranging from about 1% to about 95% (w/w) of the total composition. In another embodiment, the lactose-based diluent is present in a range of from about 50% to about 70% (w/w) of the total composition. In yet another embodiment, the lactose-based diluent is present in an amount of from 55.0% to about 56.0% (w/w) of the total composition.

The microcrystalline cellulose used in the tablet formulation of the present invention is present in amounts ranging from about 1% to about 95% (w/w) of the total composition. In another embodiment the microcrystalline cellulose is present in a range of from about 15% to about 45% (w/w) of the total composition. In yet another embodiment, the microcrystalline cellulose is present in an amount of from about 33% to about 34% (w/w) of the total composition.

The pharmaceutically acceptable disintegrants which are suitable for use in the direct compression tablets of the present invention include, but are not limited to, one or more of crospovidone, sodium starch glycolate, croscarmellose sodium, and starch. The disintegrant is present in amounts of from about 0% to about 10% (w/w) of the total composition. In another embodiment the disintegrant is present in an amount of from about 4% to about 5% (w/w) of the total composition.

The pharmaceutically acceptable glidants which are suitable for use in the direct compression tablets of the present invention include, but are not limited to, one or more of colloidal silicon dioxide and colloidal anhydrous silica. The glidant is present in amounts of from about 0.1% to about 2% (w/w) of the total composition. In another embodiment the glidant is present in an amount of from about 0.5% to about 1% (w/w) of the total composition.

The pharmaceutically acceptable lubricants which are suitable for use in the direct compression tablets of the present invention include, but are not limited to, one or more of magnesium stearate, calcium stearate, talc, and sodium stearyl fumarate. The lubricant is present in amounts of from about 0.1% to about 2% (w/w) of the total composition. In another embodiment the lubricant is present in an amount of from about 0.5% to about 1% (w/w) of the total composition.

Optionally, a pharmaceutically acceptable coating may be applied to the direct compression tablet. Conventional coating formulations, including but not limited to film formers, plasticizers, colorants, flavorings, and sweeteners, are useable in accordance with the present invention. In one embodiment of the invention, a coating composition comprising HPMC (hydroxypropyl methylcellulose), PEG (polyethylene glygol), polysorbate, and a colorant is used to coat the tablets of the invention. It is important to utilize a coating that does not adversely affect the dissolution rate of the tablet while enhancing aspects such as palatability, swallowability, and possibly shelf life.

The pharmaceutically active ingredient, galantamine and its pharmaceutically acceptable salts, is present in the direct compression tablets in a therapeutically effective amount. According to one embodiment of the invention, galantamine hydrobromide is provided as the pharmaceutically active ingredient in an amount of from about 2% to about 10% (w/w) of the total tablet composition. In another embodiment, galantamine hydrobromide is present in an amount of from about 4% to about 7% (w/w) of the total tablet composition. In yet another embodiment, galantamine hydrobromide is present in an amount of about 5% to about 6% (w/w) of the total tablet composition.

In one embodiment of the invention, direct compression tablets of the invention can comprise the following:
(a) from about 2% to about 10% (w/w) galantamine hydrobromide;
(b) from about 1% to about 95% (w/w) lactose-based diluent;
(c) from about 1% to about 95% (w/w) microcrystalline cellulose;
(d) from about 0% to about 10% (w/w) disintegrant;
(e) from about 0.1% to about 2% (w/w) glidant;
(f) from about 0.1% to about 2% (w/w) lubricant;
and optionally a coating composition.

In another embodiment, the direct compression tablets can comprise the following:
(a) from about 2% to about 10% (w/w) galantamine hydrobromide;
(b) from about 50% to about 70% (w/w) lactose anhydrous as the lactose-based diluent;
(c) from about 15% to about 45% (w/w) microcrystalline cellulose;
(d) from about 0% to about 10% (w/w) crospovidone as the disintegrant;
(e) from about 0.1% to about 2% (w/w) colloidal silicon dioxide as the glidant;
(f) from about 0.1% to about 2% (w/w) magnesium stearate as the lubricant;
and optionally a coating composition.

In yet another embodiment of the invention, the direct compression tablets can comprise the following:
(a) from about 5.0% to about 6.0% (w/w) galantamine hydrobromide;
(b) from about 5.0% to about 6.0% (w/w) lactose anhydrous;
(c) from about 33.0% to about 34.0% (w/w) microcrystalline cellulose;
(d) from about 4.0% to about 5.0% (w/w) crospovidone;
(e) from about 0.5% to about 1.0% (w/w) colloidal silicon dioxide;
(f) from about 0.5% to about 1.0% (w/w) magnesium stearate;
and optionally a coating composition.

The direct compression galantamine tablets of the invention typically contain amounts of galantamine that are therapeutically effective for treatment of conditions such as, but not limited to, Alzheimer's disease. The therapeutically effective amount of galantamine can be from about 2 mg to about 20 mg galantamine. In accordance with another embodiment of the invention, direct compression galantamine hydrobromide tablets are provided wherein the galantamine hydrobromide is present in amounts of about 4 mg, 8 mg, and 12 mg strengths. The corresponding overall tablet weights for the 4 mg, 8 mg, and 12 mg strength tablets are about 80 mg to about 120 mg (4 mg strength), about 160 mg to about 240 mg (8 mg strength), and about 240 mg to about 360 mg (12 mg strength).

The present invention also concerns a process for making the direct compression galantamine tablets as described above. In general, the process comprises the following steps:
(a) passing a galantamine active component, lactose-based diluent, microcrystalline cellulose, disintegrant (if present), and glidant (if present) through a delumping device to form a first delumped material;
(b) blending the first delumped material of step (a) to form a first blend;
(c) passing a lubricant (if present) through a delumping device;
(d) blending the delumped lubricant of step (c) with the first blend of step (b) to form a second blend;
(e) compressing the second blend into a direct compression tablet.

In an alternative embodiment, the glidant (if present) may be delumped with the lubricant (if present) in step (c) rather than in step (a).

The direct compression tablet of step (e) can then be coated with a pharmaceutically acceptable coating composition as described hereinbefore.

The tablets according to the invention may be compressed using conventional tablet compressing devices such as rotary tablet presses, such as Beta, Fette, Nova, and Hata presses. Exemplary, and not limiting, compression forces used to prepare the tablets according to the invention range when using a Fette press range from 2.5 to 5.9 kN for 4 mg, 2.9 to 7.4 kN for 8 mg, and 4.7 to 9.4 kN for 12 mg.

The delumping of the particles is done by using a pharmaceutical acceptable delumping device, such as Comil, Fitzmill, and sieve. An example of particle size after delumping as ascertained by sieve analysis is 30 mesh: 0%, 40 mesh: 0%, 60 mesh: 11%, 80 mesh: 20%, 100 mesh: 12%, 200 mesh: 28%, and pan: 30%.

The resulting tablets prepared according to the process of the invention have thickness values of, including but not limited to, from about 0.118 inches to about 0.135 inches along with hardness values of from about 2.4 kp to about 9.5 kp for the 4 mg, from about 0.154 inches to about 0.174 inches along with hardness values of from about 4.3 kp to about 13.5 kp for the 8 mg, from about 0.181 inches to about 0.199 inches along with hardness values of from about 8.4 kp to about 16.2 kp for the 12 mg. Average dissolution rate for the tablets is >85% by 30 min in 500 ml of water using USP apparatus II.

### Experimental

Tablets according to the invention were prepared as follows:

| Ingredients (per tablet) | Amount | % (w/w) |
|---|---|---|
| Galantamine hydrobromide | 5.13 mg | 5.1% |
| Microcrystalline cellulose (Avicel PH 102) | 34.00 mg | 34% |
| Lactose, NF (anhydrous) | 55.87 mg | 55.9% |
| Crospovidone, NF | 4.00 mg | 4% |
| Colloidal silicon dioxide (Cab-O-Sil) | 0.500 mg | 0.5% |
| Magnesium stearate, NF | 0.50 mg | 0.5% |
| Total: | 100.00 mg | 100% |

Processing Steps for Tablet Formulation:

The following process was used to formulate a batch of 6,700 galantamine hydrobromide tablets (100 mg each) at the 4 mg strength:

Galantamine hydrobromide (34.3 g), Avicel PH 102 (227.8 g), lactose, NF (anhydrous) (374.4 g), and crospovidone, NF (26.8 g), were delumped by passing the ingredients through a #20 mesh screen. The ingredients were then blended. The magnesium stearate (3.4 g) and Cab-O-Sil (3.4 g) were bag blended and delumped by passing the blend through a #20 mesh screen. All the delumped ingredients were then blended in a 2 L bin for 7.5 minutes at 62 rpm to form a final blend. The final blend was then compressed into tablets having a target weight of 100 mg each. This small batch was compressed on a Beta press wherein the hardness range of the resulting tablets was from 3.1 kp to 9.0 kp and the target hardness was 6.0 kp. The hardness range is a range selected for a specific formulation and process that ensures a good compression range and acceptable performance (including but not limited to friability, dissolution, and appearance) of the resulting tablet and is related to a myriad factors, including but not limited to, formulation, process, tablet weight, tablet shape, batch size, press design, and press speed.

The direct compression tablets prepared according to the above described process had thickness values of from about 0.119 inches to about 0.134 inches along with hardness values of from about 3.1 kp to about 9.0 kp. In U.S. Pat. No. 6,358,527 at column 3 lines 13-22), it was indicated that direct compression of galantamine with microcrystalline cellulose and lactose anhydrous resulted in tablets (formulations F1 and F2 of the '527 patent) which failed to meet Stage 1 dissolution specification of Q=80% after 30′ (USP 27, <711>Dissolution, pp 2303-2304, Apparatus 2 (paddle, 50 rpm)). According to the present invention, using the referenced USP standard, the average percent dissolved for the direct compression manufactured tablets of the invention was 102% at 30 minutes. This easily meets the USP specification of Q=80% at 30 minutes as described above.

In U.S. Pat. No. 6,358,527, it was also reported that with the direct blend of microcrystalline cellulose and lactose monohydrate, “A particular problem which occurred during feeding the dry blend into the tablet press for direct compression, was segregation of the tablet excipients, thus causing the tablets to have a variable composition.” (U.S. Pat. No. 6,538,527 at column 3, lines 17-20). The variability of tablets is generally measured according to the USP content uniformity method. According to the current USP (USP 27, <905> Uniformity of Dosage Units, pp 2396-2397), “the requirements for dosage uniformity are met if the amount of active ingredient in each of the 10 dosage units as determined from the Weight Variation or the Content Uniformity method lied within the range of 85.0%% to 115.0% of the label claim and the Relative standard deviation is less than or equal to 6.0%.” According to the present invention, 10 tablets prepared in accordance with the above described process were randomly selected and assayed according to the USP content uniformity method. The individual assays produced values of 98.6%, 101.4%, 99.0%, 99.7%, 99.0%, 96.6%, 98.7%, 98.4%, 98.6%, 99.7% for each of the ten tablets respectively. The relative standard deviation of the ten tablets is 1.22%. The content uniformity requirement of the USP is therefore well met by the direct compression tablets prepared according to the present invention.

In conclusion, the formulation as dislosed by the present invention has excellent content uniformity along with excellent dissolution properties.

Having described the invention in detail and by reference to preferred embodiments thereof, it will be apparent that modifications and variations are possible without departing from the scope of the invention defined in the appended claims.

1. A pharmaceutical tablet composition comprising, as an active ingredient, galantamine or a pharmaceutically acceptable salt thereof, and a pharmaceutically acceptable carrier, wherein said pharmaceutically acceptable carrier comprises a direct blend of a lactose-based diluent and microcrystalline cellulose.

2. The composition of claim 1 wherein the active ingredient is galantamine hydrobromide.

3. The composition of claim 1 further comprising one or more pharmaceutically acceptable adjuvants selected from disintegrants, glidants, lubricants, and coating agents.

4. The composition of claim 1 wherein the lactose-based diluent is present in an amount of from about 1% to about 95% (w/w).

5. The composition of claim 1 wherein the microcrystalline cellulose is present in an amount of from about 1% to about 95% (w/w).

6. The composition of claim 1 wherein the lactose-based diluent is selected from one or more of lactose anhydrous, lactose monohydrate, and spray-dried lactose.

7. The composition of claim 3 wherein the disintegrant is selected from one or more of crospovidone, sodium starch glycolate, croscarmellose sodium, and starch.

8. The composition of claim 3 wherein the glidant is selected from one or more of colloidal silicon dioxide and colloidal anhydrous silica.

9. The composition of claim 3 wherein the lubricant is selected from one or more of magnesium stearate, calcium stearate, talc, and sodium stearyl fumarate.

10. The composition of claim 1 wherein the lactose-based diluent is lactose anhydrous.

11. The composition of claim 10 wherein the lactose anhydrous is present in an amount of from about 50% to about 70% (w/w) and the microcrystalline cellulose is present in an amount of from about 15% to about 45% (w/w).

12. A pharmaceutical, direct compression, tablet composition comprising:
(a) from about 2% to about 10% (w/w) galantamine hydrobromide;
(b) from about 1% to about 95% (w/w) lactose-based diluent;
(c) from about 1% to about 95% (w/w) microcrystalline cellulose;
(d) from about 0% to about 10% (w/w) disintegrant;
(e) from about 0.1% to about 2% (w/w) glidant;
(f) from about 0.1% to about 2% (w/w) lubricant.

13. The tablet composition of claim 12 comprising:
(a) from about 2% to about 10% (w/w) galantamine hydrobromide;
(b) from about 50% to about 70% (w/w) lactose anhydrous as the lactose-based diluent;
(c) from about 15% to about 45% (w/w) microcrystalline cellulose;
(d) from about 0% to about 10% (w/w) crospovidone as the disintegrant;
(e) from about 0.1% to about 2% (w/w) colloidal silicon dioxide as the glidant;
(f) from about 0.1% to about 2% (w/w) magnesium stearate as the lubricant.

14. The tablet composition of claim 13 comprising:
(a) from about 5.0% to about 6.0% (w/w) galantamine hydrobromide;
(b) from about 5.0% to about 6.0% (w/w) lactose anhydrous;
(c) from about 33.0% to about 34.0% (w/w) microcrystalline cellulose;
(d) from about 4.0% to about 5.0% (w/w) crospovidone;
(e) from about 0.5% to about 1.0% (w/w) colloidal silicon dioxide;
(f) from about 0.5% to about 1.0% (w/w) magnesium stearate.

15. The tablet composition of claim 12 which is coated with a pharmaceutically acceptable coating agent.

16. A process of making a direct compression tablet according to claim 12 comprising the steps of:
(a) passing the galantamine hydrobromide, lactose-based diluent, microcrystalline cellulose, and disintegrant through a delumping device to form a first delumped material;
(b) blending the first delumped material of step (a) to form a first blend;
(c) passing the lubricant through a delumping device;
(d) blending the delumped lubricant of step (c) with the first blend of step (b) to form a second blend;
(e) compressing the second blend into a direct compression tablet wherein the glidant may be present in either of steps (a) or (c).

17. The process of claim 16 wherein the lactose-based diluent is selected from one or more of lactose anhydrous, lactose monohydrate, and spray-dried lactose.

18. The process of claim 16 wherein the disintegrant is selected from one or more of crospovidone, sodium starch glycolate, croscarmellose sodium, and starch.

19. The process of claim 16 wherein the glidant is selected from one or more of colloidal silicon dioxide and colloidal anhydrous silica.

20. The process of claim 16 wherein the lubricant is selected from one or more of magnesium stearate, calcium stearate, talc, and sodium stearyl fumarate.

21. The process of claim 16 wherein the tablet comprises the following:
(a) from about 2% to about 10% (w/w) galantamine hydrobromide;
(b) from about 50% to about 70% (w/w) lactose anhydrous as the lactose-based diluent;
(c) from about 15% to about 45% (w/w) microcrystalline cellulose;
(d) from about 0% to about 10% (w/w) crospovidone as the disintegrant;
(e) from about 0.1% to about 2% (w/w) colloidal silicon dioxide as the glidant;
(f) from about 0.1% to about 2% (w/w) magnesium stearate as the lubricant.

22. The process of claim 16 wherein the tablet is further coated.

23. The process of claim 21 wherein the tablet is further coated.

24. The process of claim 16 wherein the tablet of step (e) is compressed to a hardness of from about 2 kP to about 40 kP.

25. The process of claim 24 wherein the hardness is from about 4 kP to about 20 kP.

26. The process of claim 24 wherein the hardness is from about 6 kP to about 14 kP.
